(19) United States
(12) Patent Application Publication
(10) Pub. No.: US 2022/0081665 A1
(43) Pub. Date: Mar. 17, 2022

(54) Bioreactor apparatus

(71) Applicant: Patrick Power, Malden, MA (US)

(72) Inventor: Patrick Power, Malden, MA (US)

(21) Appl. No.: 17/023,152

(22) Filed: Sep. 16, 2020

(51) Int. Cl. C12M 3/06; C12M 1/00

(52) U.S. Cl. CPC C12M 27/14; C12M 29/24

(57) ABSTRACT

The present invention concerns an improved bioreactor with enhanced oxygen transfer. In lieu of the standard impeller and sparger assembly, a rotating gas sparger is used to introduce oxygen into the reactor. A mechanical drive shaft is coupled to a conduit from which a length of tubing extends. A source of pressurized gas is fluidly coupled to the tubing, which tubing is rotated via the mechanical drive shaft. The rotating tubing has a variably sized outlet for introducing oxygen bubbles into the liquid medium of the reactor, the bubbles providing both mechanical agitation and oxygen transfer due to the rotation of the tubing.

### 1. FIELD OF THE INVENTION

The present invention generally relates to an apparatus for growing or maintaining biological cells in vitro. More specifically, the present invention relates to a bioreactor apparatus and method that are effective to grow or maintain viable biological cells, while providing increased oxygen flow with low shear stress on cells.

### BACKGROUND OF THE INVENTION

Bioreactors have commonly been employed for the cultivation of living organisms, such as mammalian cells. A bioreactor includes a housing that contains cells and nutrients maintained at bioreactor conditions that permit cell growth. The growth and culture of both plant and animal cells typically require a constant supply of adequate oxygen. Oxygen diffusion in culture media is a function of a liquid-to-air surface area when operating the bioreactor. Furthermore, oxygen transfer is limited by the liquid-to-air surface area and any shear forces created by agitation and sparging, which forces cause cell destruction.

Gas sparging is required for effective oxygen transfer since introducing bubbles into a culture media by sparging results in a dramatic increase in the liquid-to-air surface area. In addition, agitation is used to break up the bubbles to thereby further increase oxygen transfer. Unfortunately, both bubbling and agitation typically have a detrimental effect on biological cells, especially mammalian cell cultures. Biological cells may be rendered non-viable through bubble breakup (due to shear stresses) and/or coalescence within the culture media, especially at a surface gas-to-liquid interface. Agitation is typically performed by one or more shaft driven impellers which can be rotated at about 400 RPM. Rotation above 400 RPM is not recommended when dealing with mammalian or other animal cells as the force produced by higher rotation speeds tends to cause destructive shear forces which can render a “batch” non-viable. Therefore, maximizing oxygen transfer in the bioreactor must be balanced by maintaining cell viability in order to increase the chances of producing a viable batch.

The present invention concerns an improved bioreactor with enhanced oxygen transfer. In lieu of the standard impeller and sparger assembly, a rotating gas sparger is used to introduce oxygen into the reactor. A mechanical drive shaft is coupled to a length of tubing. A source of pressurized gas is fluidly coupled to the tubing, which tubing is rotated via the mechanical drive shaft. The tubing has apertures to allow the passage of pressurized gas through the point of least resistance through the spinning tubing and down to the liquid in the bioreactor. The pressurized gas is connected to the outside housing of the device by means of a quick connect locking device. In larger “sterilize in place” bioreactors the pressurized gas can be mechanically attached. The rotating tubing has a variably sized outlet for introducing oxygen bubbles into the reactor, the bubbles providing both mechanical agitation and oxygen transfer due to the rotation of the tubing.

### SUMMARY OF THE INVENTION

It is a major object of the invention to provide an improved bioreactor apparatus.

It is another object of the invention to provide an improved bioreactor apparatus which produces enhanced oxygen transfer without destructive impeller forces.

It is another object of the invention to provide an improved bioreactor apparatus which provides both oxygenation and mechanical agitation in a single device.

It is another object of the invention to provide an improved bioreactor apparatus which requires less energy than standard bioreactors.

It is another object of the invention to provide an improved bioreactor apparatus which requires only a single inlet into the reactor vessel for both agitation and oxygenation to reduce the possibility of contamination.

It is another object of the invention to provide an improved bioreactor apparatus that can be scaled up for use in vessels from 1 to 20,000 liters and larger.

It is another object of the invention to provide an improved bioreactor apparatus that can control bubble size at the gas liquid interface.

### BRIEF DESCRIPTION OF THE DRAWINGS

FIG. 1 shows a cross section of the bioreactor apparatus of the invention.

FIG. 2 shows a perspective view of the bioreactor apparatus of the invention.

FIG. 3 shows a cross section of the bioreactor of FIG. 1 with microprocessor controlled components installed.

FIG. 4 shows how the apparatus works in a typical bioreactor setup.

### DETAILED DESCRIPTION

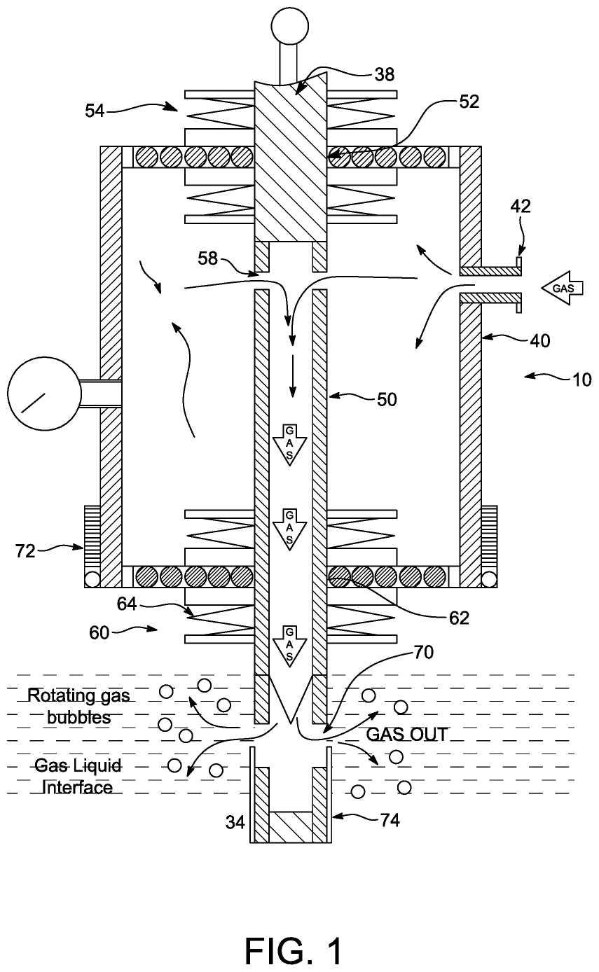

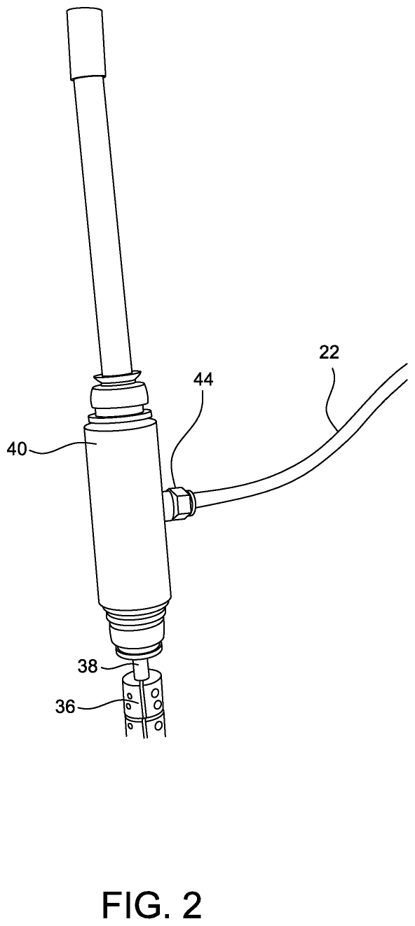

The present invention is directed to a bioreactor apparatus which provides enhanced oxygen transfer within a liquid culture media. Referring now to FIGS. 1-4 the apparatus, generally indicated by the numeral 10, is designed to be disposed entirely within a bioreactor vessel 20 with oxygen supplied via a length of tubing 22 fluidly coupled to a supply of pressurized oxygen 44. It should be noted here that the apparatus 10 can be scaled up or down for use in vessels 20 ranging in size from 1 to 20,000 liters or more, or from experimental to industrial size. Also, the apparatus 10 can be used with the so called disposable vessels.

With particular reference to FIGS. 1-3, it can be seen that the apparatus 10 has an elongated, generally tubular main body 40 having upper 32 and lower ends 34. The upper end 32 has a motor 36 attached thereto, the motor mechanically coupled to a drive shaft 38 so that motor 36 can apply rotating motive power to the shaft 38 and tubing 50, the tubing 50 having a short length of same diameter rigid conduit for attachment to the shaft 38. A generally cylindrical sealed gas inlet enclosure 40 surrounds the upper portion of the apparatus 10 and includes an inlet port 42 designed for fluid tight coupling to a conduit such as flexible tubing 22 which is coupled to a source of pressurized gas 44 (usually air or oxygen in the present case). The gas may also be an air/oxygen mix or CO2 mix. Ph control may be attained by adjusting the gas mix, and N2 may be used for deoxygenation. The rate of gas flow, as well as the mixture is controlled by a multiplexing valve arrangement or mass flow controllers 80 connected between multiple gas sources and inlet port 42. The valves 80 may be manually adjusted or automatically adjusted by controller 90 as desired to achieve a predetermined gas flow.

A key aspect of the invention is that the apparatus 10 provides both sparging (gas introduction) and mechanical agitation without the use of impellers or other known agitation devices or techniques. The apparatus 10 is essentially a combination of an impeller and a sparger which operates to introduce oxygen and mechanical agitation to the liquid media without generating damaging shear forces which can destroy especially animal cells and effectively reduce the number of viable batches produced by the bioreactor. To that end, a length of tubing 50 is attached in sealing engagement as by glue or adhesive to the lower end of drive shaft 38 for disposable units, so as to rotate therewith. Alternatively, the tubing 50 may be permanently molded onto the lower end of drive shaft 38. In either case, the tubing 50 is substantially rigid. For stainless steel reactors the tubing 50 is attached by welding or other permanent means. The lower portion of shaft 38 extends into and through enclosure 40 via orifice 52, the shaft 38 surrounded by a sealing member 54, which member 54 is comprised of a sealed bearing and seals attached to enclosure 40. An upper portion of the tubing 50 has at least one opening 58 formed therein to allow gas to flow through the tubing 50. The number and size of openings 58 may be varied to create a desired flow rate of oxygen through the tube 50 as would be apparent to one of skill in the art. The lower end 60 of the enclosure 40 includes a second orifice 62 and corresponding seal 64, the orifice axially aligned with orifice 52 so that tubing 50 is approximately centrally positioned within. Thus, the shaft 38 and tubing 50 are sealingly engaged within enclosure 40 so that pressurized gas may flow into and through inlet port 42, into opening 58 (which is rotating when the apparatus 10 is in operation) and out through outlet 70.

The apparatus 10 is suspended within vessel 20 as by a bracket 72 or other suitable means so that it remains stationary when in use. As shown the bracket 72 can be mechanically attached to the center of the head plate of the reactor vessel 20 screws or other fastening members, with a sealing O-ring. This allows for easy removal of the apparatus 10, bracket, etc. for servicing and maintenance, while allowing for containment of contamination risk.

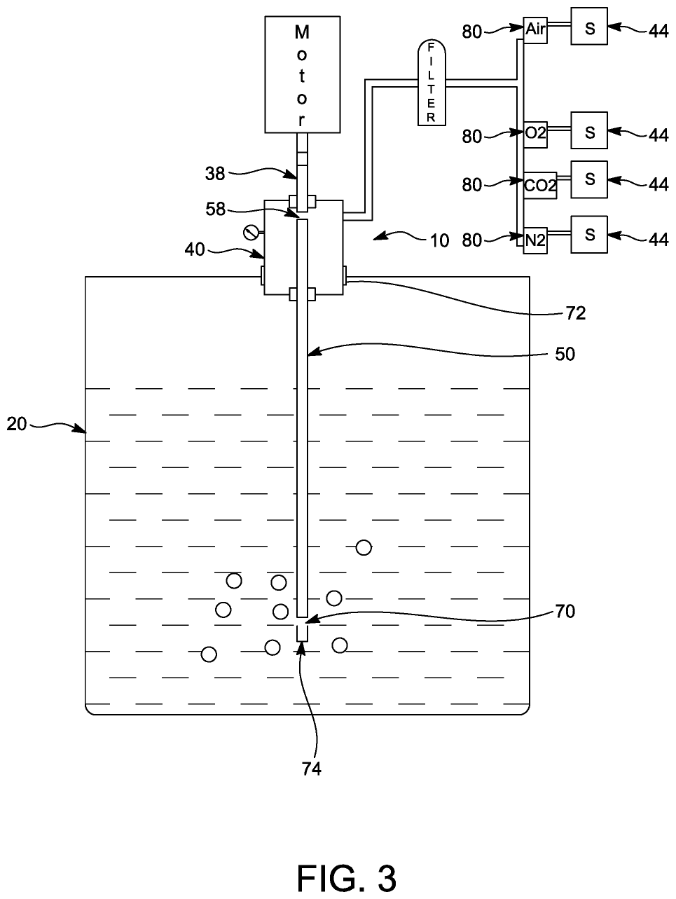

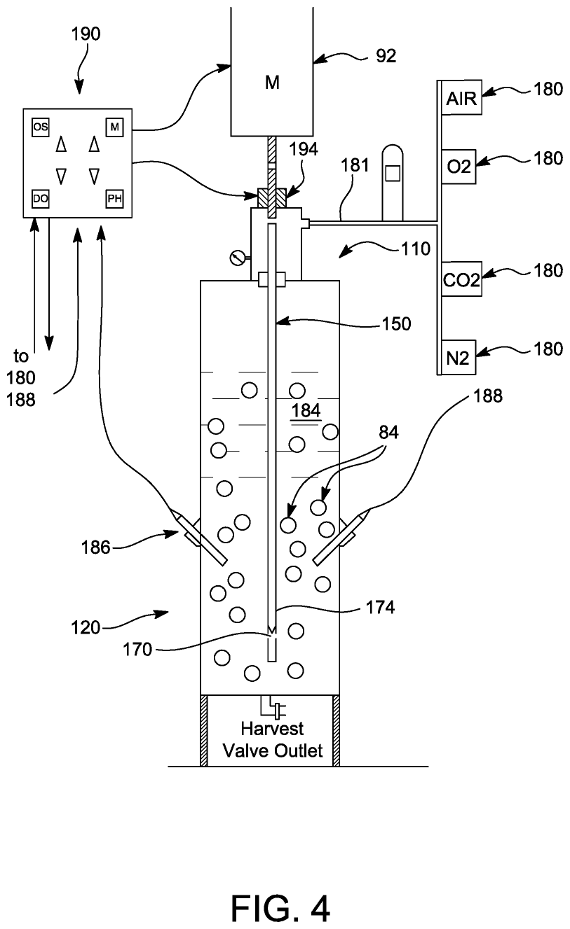

Referring now particularly to FIG. 3, the vessel 20 of FIG. 1 including a mass flow controller 80 and a controllable electrical valve 74 is shown. The mass flow controller 80 and electrical valve 74 allow for more control over the amount and type of gas applied to the reaction, as explained in more detail below with reference to the embodiment of FIG. 4. It can be appreciated that a valve 74 allows users to control both the size of gas bubbles and the rate of gas flow.

Referring now particularly to FIG. 4, a modified embodiment 100, particularly useful for larger vessels, is shown. Outlet 170 has a variable opening as determined by component 174, which may be a motorized threaded occlusion member which allows more or less gas flow depending upon position as in the prior embodiment. Other mechanisms for automatically or manually varying the size/diameter of the opening of outlet 170 may be used as would be apparent to one of skill in the art. Component 174 may be adjustably controlled exteriorly of the vessel 120 using means that would be apparent to one of skill in the art. For example, an electric motor 194 may be operably linked to component 174 so that the component 174 can be used to remotely control bubble size by varying the opening size of outlet 170. The gas rate or gas volume input per unit time can be controlled by mass flow controller 180 to increase the amplitude (i.e., number and size of bubbles of a given size per unit time) as will be explained below. The mass flow controller 180 receives gas from several sources of pressurized gases and allows gas flow into and through conduit 181 which may include a filter for impurities. This functionality is necessary since different cells have different oxygen uptake rates (OUR), which necessitates control of the oxygen transfer rate (OTR) by controlling the bubble size and amplitude.

A controller and associated control panel 190 allow a user to interface with the digitally controllable components of the system. In addition to motor 194, the controller 190 controls the operation of motor 92, the valve positions of mass flow controller 180, and monitors output signals from the sensors 186, 188. Feedback from dissolved oxygen probe 186, which senses the oxygenation of the liquid media contained within the vessel, is sent to controller 190 which determines the bubble size and amplitude by adjusting the output of mass flow controllers 180 to a desired flow rate to satisfy the desired dissolved oxygen set point. The speed of motor 92 can be controlled by controller 190 to a speed higher than 400 RPM, which is higher than traditional stirred tank bio-reactors. A higher stirring rate allows for more options when seeking ideal conditions for cell growth for different types of cells. Control of ph level of the liquid in the bioreactor vessel 120 is facilitated by feedback from ph probe 188 which sends a signal to controller 190 regarding the instant ph level. The flow rate of CO2 via mass flow controllers 180 may be adjusted in accordance with the signal from probe 188.

In larger “sterilize in place” reactors such as that shown in FIG. 4 it is necessary to install a check valve 176 inside tubing 150 due to the buildup of pressure in the vessel 120 during sterilization.

In use, once the apparatus 10, 100 is positioned within the bioreactor vessel, the motor 36, 92 may be started to cause rotation of the shaft and tubing 50, 150 causing rotating bubbles to be injected into the bottom of the vessel 20, 120. Both the rate of stirring and the amount and size of bubbles can be automatically monitored and controlled as explained above.

I claim:

1. An apparatus for applying gas and mechanical agitation to a bioreactor vessel comprising:
an generally elongated main body having top and bottom ends, said top end having a motor affixed thereto and said bottom end having an outlet;
a motor affixed to said top end and coupled to a length of tubing, said length of tubing terminating at said outlet and having an aperture formed therein, said motor providing rotation to said tubing;
a source of pressurized gas selectively connected to said apparatus; whereby said motor can cause rotation of said tubing and said source of gas can cause gas to flow out of said outlet.

2. The apparatus of claim 1 wherein said outlet has a opening with a variable diameter to control gas bubble size.

3. The apparatus of claim 2 wherein the diameter of said opening may be automatically adjusted.

4. The apparatus of claim 2 wherein the diameter of said opening is adjusted in accordance with control signals from a feedback loop.

5. The apparatus of claim 4 including a sensor for determining the gas bubble size generated at said outlet, said sensor generating the control signals.

6. The apparatus of claim 1 wherein said motor rotates at a speed of greater than 400 RPM.

7. The apparatus of claim 1 including a controller to control rotation speed of said motor in response to a control signal.

8. The apparatus of claim 1 wherein said gas flows out at a predetermined rate, said rate determined by feedback control signals generated in response to sensed oxygenation rates.
